(19) United States
(12) Patent Application Publication
(10) Pub. No.: US 2013/0296876 A1
(43) Pub. Date: Nov. 7, 2013

(54) CYSTOTOME DEVICE

(71) Applicant: Carl Rea Jordan, Folsom, CA (US)

(72) Inventor: Carl Rea Jordan, Folsom, CA (US)

(21) Appl. No.: 13/886,913

(22) Filed: May 3, 2013

Related U.S. Application Data
(60) Provisional application No. 61/642,233, filed on May 3, 2012.

(51) Int. Cl. A61F 9/007

(52) U.S. Cl. CPC A61F 9/00745
USPC 606/107

(57) ABSTRACT

A cystotome device that includes a cystotome loop utilized to ablate one or more cataracts and a shaft, the cystotome loop coupled to a first end of the shaft and a shaft connection housing coupled to a second end of the shaft. The cystotome device also includes a hand piece body, a first end of the hand piece body coupled to the shaft connection housing, wherein the hand piece body is grasped by a user to maneuver and position the cystotome device. The cystotome device also includes one or more piezoelectric crystals disposed within the hand piece body, the one or more piezoelectric crystals produces ultrasonic waves directed and applied to the one or more cataracts and a power supply cord coupled to the second end of the hand piece body, the power supply cord providing an electrical conduit of electrical power to power the one or more piezoelectric crystals.

This application claims priority to U.S. Provisional Application 61/642,233 filed on May, 3, 2012, the entire disclosure of which is incorporated by reference.

### BACKGROUND OF THE INVENTION

1. Field of the Invention

The present invention is a cataract surgery device. More specifically, the present invention is a cystotome device.

2. Description of the Related Art

Of approximately 3,000,000 cataract surgeries demanding capsulotomies, typically the cataract surgeries utilize a manual bent needle capsulorhexis assisted with forceps, to remove a patient's lens capsule. which results in a meticulously slow and demanding aspect of cataract surgery. Utilization of the capsulorhexis method has a slow learning curve with a danger of capsular tearing resulting in a failed surgical procedure.

Cataract surgeons must access the cataractous lens to remove it. If the patient's lens capsule can be more easily and safely removed, the cataract surgeries will be faster and safer with improved results. Every cataract patient benefits by a shorter time on the operating table resulting in lower infection rates.

### BRIEF SUMMARY OF THE INVENTION

The present invention is a cataract surgery device. More specifically, the present invention is a cystotome device.

The cystotome device includes a cystotome loop utilized to ablate one or more cataracts and a shaft, the cystotome loop coupled to a first end of the shaft and a shaft connection housing coupled to a second end of the shaft. The cystotome device also includes a hand piece body, a first end of the hand piece body coupled to the shaft connection housing, wherein the hand piece body is grasped by a user to maneuver and position the cystotome device. The cystotome device also includes one or more piezoelectric crystals disposed within the hand piece body, the one or more piezoelectric crystals produces ultrasonic waves directed and applied to the one or more cataracts and a power supply cord coupled to the second end of the hand piece body, the power supply cord providing an electrical conduit of electrical power to power the one or more piezoelectric crystals.

The utilization of the cystotome device is relatively faster than performing traditional capsulorhexis, taking approximately in the range of 3 to 4 seconds to utilize the cystotome device versus approximately two minutes for performing traditional capsulorhexis. The cystotome device utilizes a 360 degree, continuous and curvilinear loop. Capsulorhexis will be routinely irregular and very rarely continuous. The capsulorhexis technique typically requires hours of practice. The cystotome device can be utilized after seeing only one procedure utilized with the cystotome device.

It is an object of the present invention to provide a cystotome device that shortens a surgeons' time performing a cataract surgery and eliminating capsulorhexis.

It is an object of the present invention to provide a cystotome device that allows a surgeon to perform more cataract surgeries per day.

It is an object of the present invention to provide a cystotome device that results in a relatively safer, easier and quicker cataract surgical procedure than performing a traditional capsulorhexis.

### BRIEF DESCRIPTION OF THE DRAWINGS

The present invention will be described by way of exemplary embodiments, but not limitations, illustrated in the accompanying drawings in which like references denote similar elements, and in which:

FIG. 1 illustrates a side view of a cystotome device, in accordance with one embodiment of the present invention.

FIG. 2 illustrates a front view of a cystotome device in a plurality of positions, in accordance with one embodiment of the present invention.

FIG. 3 illustrates a side view of a cystotome loop of a cystotome device, in accordance with one embodiment of the present invention.

### DETAILED DESCRIPTION OF ILLUSTRATIVE EMBODIMENTS

Various aspects of the illustrative embodiments will be described using terms commonly employed by those skilled in the art to convey the substance of their work to others skilled in the art. However, it will be apparent to those skilled in the art that the present invention may be practiced with only some of the described aspects. For purposes of explanation, specific numbers, materials and configurations are set forth in order to provide a thorough understanding of the illustrative embodiments. However, it will be apparent to one skilled in the art that the present invention may be practiced without the specific details. In other instances, well-known features are omitted or simplified in order not to obscure the illustrative embodiments.

Various operations will be described as multiple discrete operations, in turn, in a manner that is most helpful in understanding the present invention, however, the order of description should not be construed as to imply that these operations are necessarily order dependent. In particular, these operations need not be performed in the order of presentation.

The phrase “in one embodiment” is used repeatedly. The phrase generally does not refer to the same embodiment, however, it may. The terms “comprising”, “having” and “including” are synonymous, unless the context dictates otherwise.

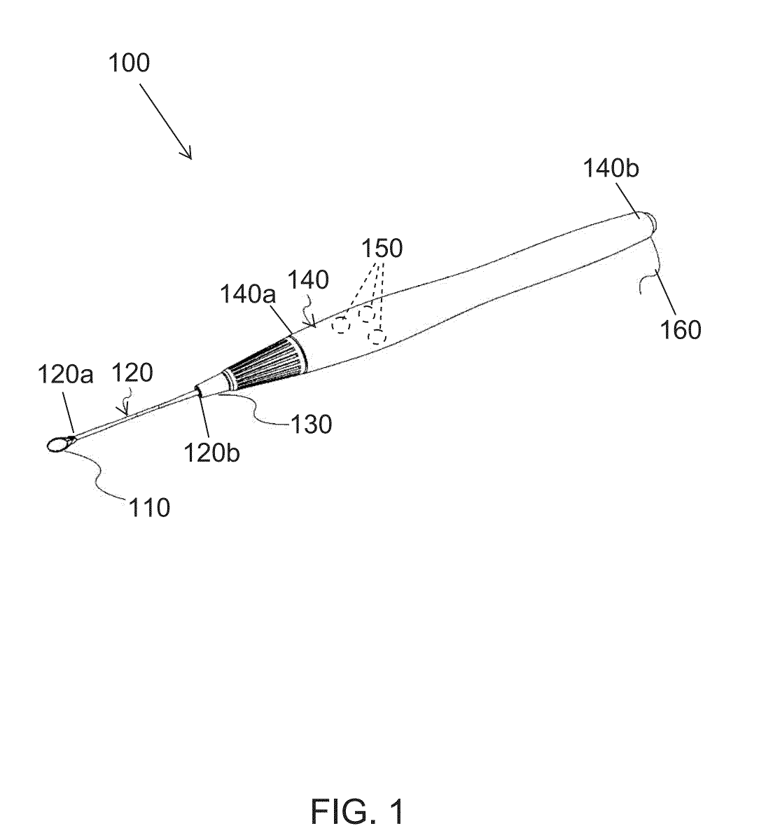

FIG. 1 illustrates a side view of a cystotome device 100, in accordance with one embodiment of the present invention.

The cystotome device 100 can have a cystotome loop 110, a shaft 120, a shaft connection housing 130, a hand piece body 140, one or more piezoelectric crystals 150 and a power supply cord 160. The cystotome loop 110 can be utilized to ablate one or more cataracts from an eye or be utilized for any other suitable use. The eye can be a human eye, an animal eye or other suitable type of eye. The cystotome loop 110 can have a diameter in the range of 3 mm to 6 mm or any other suitable diameter. The shaft 120 can have a first end 120a and a second end 120b. The cystotome loop 110 can be coupled to the first end 120a of the shaft 120. The shaft connection housing 130 can be coupled to the second end 120b of the shaft 120. The hand piece body 140 can have a first end 140a that can be coupled to the shaft connection housing 130. The hand piece body 140 can be grasped by a user to maneuver and position the cystotome device 100 to perform cataract surgery or to perform any other suitable procedure such as a cardiovascular procedure, an ontological procedure or a nasopharngeal procedure. The one or more piezoelectric crystals 150 can be disposed within the hand piece body 140. FIG. 1 illustrates three piezoelectric crystals 150, although any suitable number of the one or more piezoelectric crystals 150 can be utilized. The one or more piezoelectric crystals 150 can produce ultrasonic waves that can be directed and applied to an eye area while the cystotome device 100 is being utilized. The power supply cord 160 is coupled to a second end 140b of the hand piece body 140. The power supply cord 160 provides an electrical conduit of electrical power to power the one or more piezoelectric crystals 150. The power supply cord 160 can supply electrical power to the one or more piezoelectric crystals 150 from any suitable power source such as an electrical outlet or a battery.

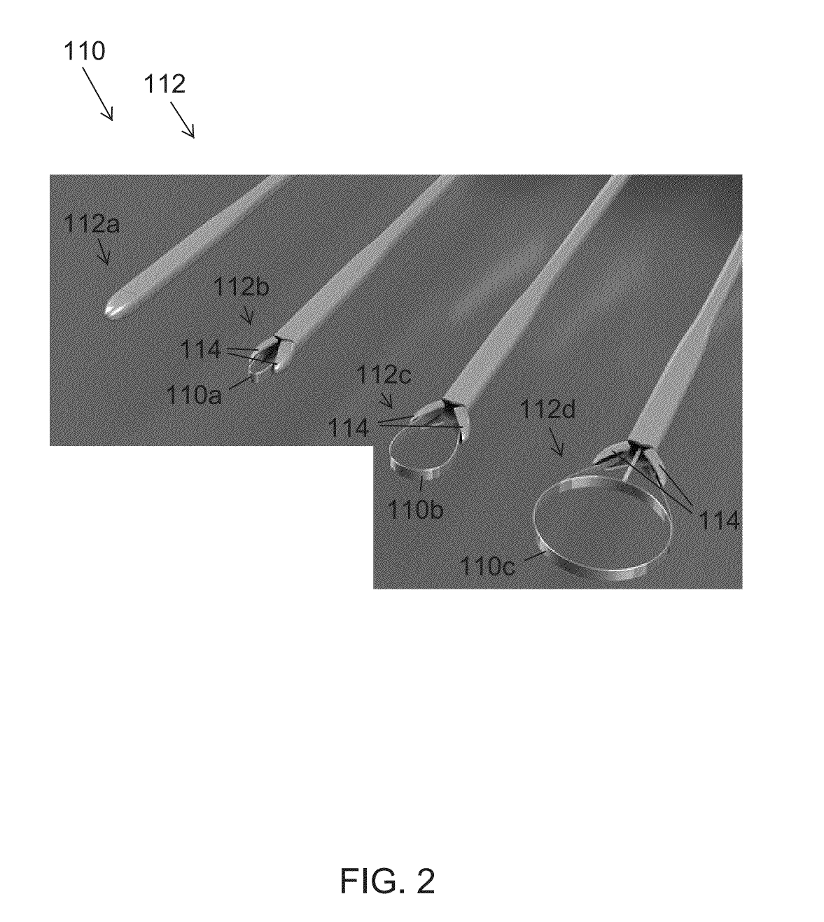

FIG. 2 illustrates a front view of a cystotome loop 110 in a plurality of positions 112, in accordance with one embodiment of the present invention. The positions 112 include a first position 112a, a second position 112b, a third position 112c and a fourth position 112d. The first position 112a can be the cystotome loop 110 in a fully closed position. The second position 112b can be where a pair of tip halves 114 can be partially separated and allow a closed cystotome loop 110a to extend through the pair of tip halves 114. The pair of tip halves 114 can be made of aluminum, steel, titanium and can be computer numerical controlled or CNC lathe tuned to a range of resonant frequencies. The third position 112c can be where the pair of tip halves 114 can be fully separated and a partially open cystotome loop 110b can extend through the fully separated pair of tip halves 114. The fourth position 112d can be where the pair of tip halves 114 can be fully separated and a fully open cystotome loop 110c can extend through the fully separated pair of tip halves 114.

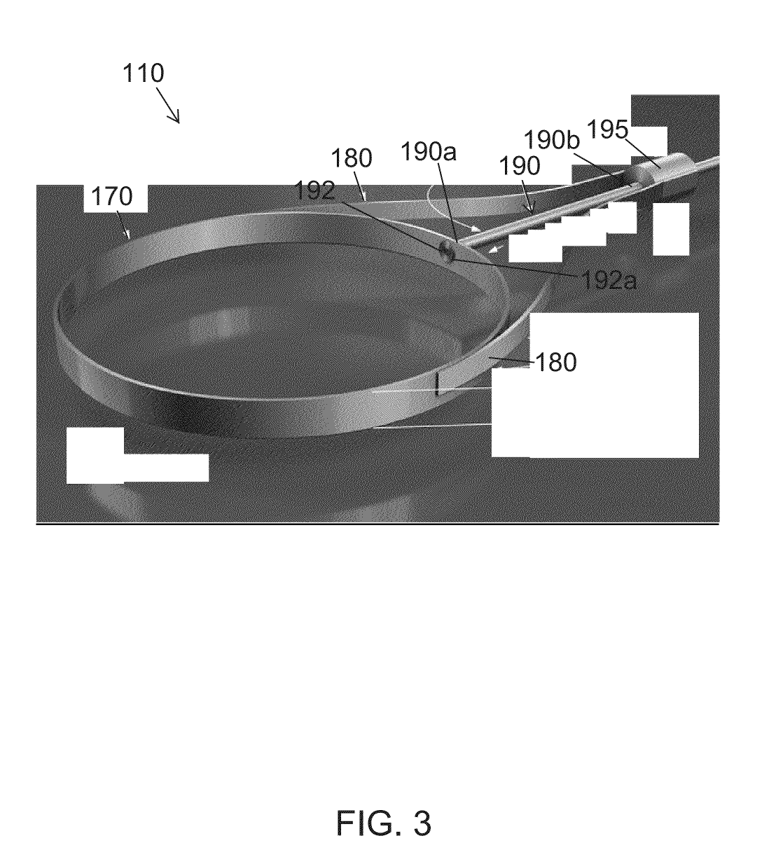

FIG. 3 illustrates a side view of a cystotome loop 110 of a cystotome device 100, in accordance with one embodiment of the present invention. The cystotome loop 110 illustrated in FIG. 3 is a fully open cystotome loop 110c.

The cystotome loop 110 can have a circular blade 170, a pair of supports 180, an actuator rod 190 and a guide 195. The circular blade 170 can be a relatively thin and flexible blade made of stainless steel such as 316 stainless steel or any other suitable material. The circular blade 170 can also be a nitinol memory wire or a nitinol memory band each with a small bending radius. The circular blade 170 can be approximately 0.5 mm wide or any other suitable width. The pair of supports 180 can be coupled to each side of the cystotome loop 110 to support the cystotome loop 110. The actuator rod 190 can have a first end 190a and a second end 190b. The first end 190a of the actuator rod 190 can be coupled to a back of the cystotome loop 110 by a fastener 192 such as a rivet 192a, a bolt (not shown) or other suitable fastener. The actuator rod 190 can extend the cystotome loop 110. The guide 195 can be coupled to the second end 190b of the actuator rod 190. The guide 195 can extend the actuator rod 190 to extend the cystotome loop 110.

Cataract surgery enables entry via a small incision (approximately less than 1.5 mm) into an anterior chamber of an eye. The cystotome device can allow continuous, curvilinear capsulotomy of approximately 5 mm in diameter or greater. The cystotome device can serve as a cannula-like probe, with an outside diameter of less than approximately 1.47 mm that can house a compressed circle within a lumen with an approximate inside diameter of 1.067 mm. The cystotome loop can be a relatively thin, flexible, 316 stainless steel or alternately a nitinol memory wire or a nitinol memory band with a small bending radius. The cystotome loop can have a flattened band of stainless steel which has been milled to enhance compressibility. Within the anterior chamber, the compressed metallic band or wire can be opened to the full diameter of approximately 5 mm. Ultrasonic energy in the range of 20 KhZ to 60 KhZ can be tuned to resonate with the approximate 5 mm cystotome loop. An approximate 5 mm capsulotomy can be performed with the cystotome device in less than approximately 3 to 4 seconds. The cystotome device can pass an approximate 5 mm cystotome readily through an approximate 1.5 mm incision.

The cystotome device can provide a method to perform a surgical capsulotomy preparatory to remove one or more cataracts. Specifically, state-of-the-art cataract surgery can demand the smallest possible clear corneal incision (e.g., less than 1.5 mm) with the shortest time surgical procedure, a capsulotomy that must be continuous and curvilinear forming a circle, that reduces vitreous loss and has a relatively fast cortical clean-up.

Entry into the anterior chamber of an eye must be unrestricted which can be achieved with the cystotome device. The cystotome device can have a relatively smooth torpedo-shaped tip. The cystotome device can be fully expandable and can have a full 360 degree, approximately 5 mm diameter cystotome ring. Ultrasonic energy in the range of 20 kHz to 60 kHz can be delivered to the cutting ring by a set of calibrated piezoelectric crystals conducting or inducting energy to the cystotome device. The cystotome device can provide a surgical technique to make a continuous, curvilinear capsulotomy in approximately less than 5 seconds. A pair of tip halves can allow the cystotome ring to protrude from a lumen of a cannula-like needle. The cystotome ring can be made of a collapsible material such as nitinol or stainless steel. The pair of tip halves can be securely attached by a plurality of fine screw threads to the actuator rod inside the hand piece body. The pair of tip halves can be fabricated from aluminum, steel and, in some cases, titanium and a CNC lathe tuned to a range of resonant frequencies. Three or more piezoelectric crystals can be provided that are energized by a separate power supply. At the extreme proximal termination, a classical ultrasound, male connector can provide input connection for a power source such as an electrical outlet, a battery or other suitable power source.

Cataract surgery can be done in a very small anatomical section of the eye called the anterior chamber. The ceiling of the anterior chamber, the most posterior layer of the cornea, which is the endothelium, is a single cell layer subject to serious damage if not operatively managed skillfully. The cystotome device must be minuscule to fit into an approximately less than 1.5 mm initial corneal incision. However, once in the anterior chamber, the cystotome device must expand to rapidly perform an optimum size capsulotomy. The cystotome device can provide a range of cystotome diameters, e.g., in the range of approximately 3 mm to 5.5 mm or approximately 6 mm to special order. While the present invention has been related in terms of the foregoing embodiments, those skilled in the art will recognize that the invention is not limited to the embodiments described. The present invention can be practiced with modification and alteration within the spirit and scope of the appended claims. Thus, the description is to be regarded as illustrative instead of restrictive on the present invention.

What is claimed is:

1. A cystotome device, comprising:
a cystotome loop utilized to ablate one or more cataracts;
a shaft having a first end and a second end, the cystotome loop coupled to the first end of the shaft;
a shaft connection housing coupled to the second end of the shaft;
a hand piece body having a first end and a second end, the first end of the hand piece body coupled to the shaft connection housing, wherein the hand piece body is grasped by a user to maneuver and position the cystotome device;
one or more piezoelectric crystals disposed within the hand piece body, the one or more piezoelectric crystals produces ultrasonic waves directed and applied to the one or more cataracts; and
a power supply cord coupled to the second end of the hand piece body, the power supply cord providing an electrical conduit of electrical power to power the one or more piezoelectric crystals.

2. The cystotome device according to claim 1, wherein the cystotome loop is in a plurality of positions that include a first position, a second position, a third position and a fourth position.

3. The cystotome device according to claim 2, wherein the first position includes the cystotome loop in a fully closed position.

4. The cystotome device according to claim 2, wherein the second position includes a pair of tip halves that are partially separated and allow the closed cystotome loop to extend through the pair of tip halves.

5. The cystotome device according to claim 4, wherein the third position includes the pair of tip halves being fully separated and having a partially open cystotome loop extending through the fully separated pair of tip halves.

6. The cystotome device according to claim 4, wherein the fourth position includes the pair of tip halves being fully separated and a fully open cystotome loop extending through the fully separated pair of tip halves.

7. The cystotome device according to claim 4, wherein the pair of tip halves is made of material selected from the group consisting of aluminum, steel or titanium.

8. The cystotome device according to claim 1, wherein the cystotome loop has a diameter in the range of 3 mm to 6 mm.

9. A cystotome device, comprising:
a cystotome loop having a circular blade, a pair of supports, an actuator rod and a guide, the cystotome loop utilized to ablate one or more cataracts, the pair of supports coupled to each side of the cystotome loop to support the cystotome loop, the actuator rod having a first end and a second end, the first end of the actuator rod coupled to a back of the cystotome loop by a fastener, wherein the actuator rod extends the cystotome loop, the guide coupled to the second end of the actuator rod and the guide extends the actuator rod to extend the cystotome loop;
a shaft having a first end and a second end, the cystotome loop coupled to the first end of the shaft;
a shaft connection housing coupled to the second end of the shaft;
a hand piece body having a first end and a second end, the first end of the hand piece body coupled to the shaft connection housing, wherein the hand piece body is grasped by a user to maneuver and position the cystotome device;
one or more piezoelectric crystals disposed within the hand piece body, the one or more piezoelectric crystals produces ultrasonic waves directed and applied to the one or more cataracts; and
a power supply cord coupled to the second end of the hand piece body, the power supply cord providing an electrical conduit of electrical power to power the one or more piezoelectric crystals.

10. The cystotome device according to claim 9, wherein the cystotome loop is in a plurality of positions that include a first position, a second position, a third position and a fourth position.

11. The cystotome device according to claim 10, wherein the first position includes the cystotome loop in a fully closed position.

12. The cystotome device according to claim 10, wherein the second position includes a pair of tip halves that are partially separated and allow the closed cystotome loop to extend through the pair of tip halves.

13. The cystotome device according to claim 12, wherein the third position includes the pair of tip halves being fully separated and having a partially open cystotome loop extending through the fully separated pair of tip halves.

14. The cystotome device according to claim 12, wherein the fourth position includes the pair of tip halves being fully separated and a fully open cystotome loop extending through the fully separated pair of tip halves.

15. The cystotome device according to claim 12, wherein the pair of tip halves is made of material selected from the group consisting of aluminum, steel or titanium.

16. The cystotome device according to claim 9, wherein the cystotome loop is a selected one of a thin and flexible blade and a thin and flexible band.

17. The cystotome device according to claim 16, wherein the cystotome loop is made of material selected from the group consisting of stainless steel, 316 stainless steel or nitinol.

18. The cystotome device according to claim 9, wherein the cystotome loop has a diameter in the range of 3 mm to 6 mm.

19. The cystotome device according to claim 9, wherein the circular blade is 0.5 mm wide.

20. The cystotome device according to claim 9, wherein the fastener is a rivet.
